(19) United States
(12) Patent Application Publication
(10) Pub. No.: US 2026/0015607 A1
(43) Pub. Date: Jan. 15, 2026

(54) SPATIALLY LAYERED DNA STORAGE METHOD FOR LARGE-SCALE OLIGO POOLS

(71) Applicant: TIANJIN UNIVERSITY, Tianjin (CN)

(72) Inventors: Weigang CHEN, Tianjin (CN); Yingjin YUAN, Tianjin (CN); Yuxin ZHANG, Tianjin (CN); Qi GE, Tianjin (CN); Haoran ZHANG, Tianjin (CN); Quan GUO, Tianjin (CN)

(21) Appl. No.: 19/045,141

(22) Filed: Feb. 4, 2025

(30) Foreign Application Priority Data
Jul. 11, 2024 (CN) 2024109275215

(51) Int. Cl. C12N 15/10; G06F 18/2433; H03M 13/13

(52) U.S. Cl. CPC C12N 15/1065; G06F 18/2433; H03M 13/13

(57) ABSTRACT

The present disclosure discloses a spatially layered DNA storage method for large-scale oligonucleotide pools, employing a DNA spatially layered coding method to enable real-time data readout; the unordered DNA strands are spatially organized into an addressable base array, and the live data are encoded chronologically into sequential coding layers, wherein bases are mapped to crosscutting identical positions across all strands; for recovery, a live and accelerated approach to spatially form a coding layer is provided, and the error correction codes are utilized to fill the base gap, enabling continuous, real-time streaming; a layer-wise spatial-temporal recovery method is presented to facilitate an error-free data stream, spatially achieving instant consensus of multiple signals within a layer, and temporally updating flow signals via the previous successfully decoded layers; the error correction and readout methods provided by the present disclosure can match the sequencing process, achieving simultaneous sequencing and real-time decoding.

### CROSS-REFERENCE TO RELATED APPLICATION

This application claims priority from the Chinese patent application 2024109275215 filed Jul. 11, 2024, the content of which is incorporated herein in the entirety by reference.

### TECHNICAL FIELD

The present disclosure relates to the technical field of deoxyribonucleic acid (DNA) storage, and in particular, to a DNA storage method based on spatially layered large-scale oligo pools for achieving rapid data readout by using high-throughput synthetic oligo pools.

### BACKGROUND ART

With the rapid development of global information technology, the data amount presents explosive growth, and synthetic deoxyribonucleic acid has become a promising medium for archival data storage having high storage density and long-term persistence. Compared with existing storage media using magnetic, optical and electrical media, DNA serving as a data storage medium has the characteristics of small volume, large density, long-lasting information retention time, etc. For example, in terms of density, Erlich et al. confirmed that the storage density of DNA can reach 215 PB/g (DNA Fountain enables a robust and efficient storage architecture. Science 355, 950-954, 2017), researchers also confirmed that the data storage density can reach 125 PB/g in molecular pools having larger storage scale and more complex reading. In terms of storage stability, Song et al. proved through accelerated aging tests that information stored in DNA can be stored for thousands of years at room temperature in a laboratory (Robust data storage in DNA by de Bruijn graph-based de novo strand assembly. Nat. Commun. 13, 5361, 2022). And Grass et al. confirmed that information-encoded DNA can be stored for thousands of years if stored in silica (Robust chemical preservation of digital information on DNA in silica with error-correcting codes. Angew. Chem. Int. Ed. 54, 2552-2555, 2015).

With the development of high-throughput DNA synthesis and sequencing technology, the use of large numbers of oligo pools for data storage has become an important mode for DNA data storage. By using the oligo pools as a storage medium, large-scale data writing can be achieved by means of a high-throughput synthesizer. In this mode, data is encoded, decomposed and distributed into a large number of DNA strands. In addition, to meet practical data storage standards, efficient encoding methods for error correction, such as digital fountain codes and Reed-Solomon (RS) codes, have been integrated. Erlich et al. used fountain codes and RS codes for solving the problem of sequence loss, achieving ultra-high density DNA storage. Grass et al. used two rounds of orthogonal Reed-Solomon (RS) error correction codes to achieve lossless recovery of original data. Press et al. developed a concatenated code encoding scheme, wherein an inner code is Hash Encoded, Decoded by Greedy Exhaustive Search (HEDGES), for correcting insertion and deletion errors, and an outer code is an RS code, for correcting a residual error (HEDGES error-correcting code for DNA storage corrects indels and allows sequence constraints. Proc. Natl. Acad. Sci. U.S.A. 117, 18489-18496, 2020).

However, in the data storage mode based on oligo pools, there are still challenges such as data writing/reading processes having high synthesis costs and time consuming. During data read-out, next-generation high-throughput sequencing such as Illumina is generally used, and is based on a sequencing-by-synthesis technology, but the sequencing process thereof is time-consuming.

When the next-generation high-throughput sequencing is used, current sequencing technologies generally detect single nucleotides (or homopolymeric strands) in one run. Only after all nucleotides in each single strand are retrieved, can encoded data be recovered, which generally takes at least a few hours. In high-throughput sequencing technologies, an Ion Torrent sequencing technology is a sequencing-by-synthesis technology that uses a semiconductor chip as a carrier, and converts chemical signals into electrical signals by detecting a change in PH caused by H+ released by DNA strands during synthesis, to acquire base information. The Ion Torrent sequencing technology carrying the semiconductor chip is simpler, faster, more cost-effective, and more scalable.

Meanwhile, errors may occur during nucleotide synthesis or sequencing. Since DNA sequencing data is generally analyzed based on the entire strand, most DNA storage schemes, including the encoding process, are configured to use the entire strand as a whole. Base insertion/deletion may disrupt the strand, and conventional error correction codes may not function efficiently. Base insertion/deletion errors become a very challenging problem in DNA data storage. In Ion Torrent sequencing, non-terminated polymerization makes it difficult to accurately count merging events in homopolymer DNA. Thus, base insertion or deletion dominates sequencing errors. In conclusion, the strand-based data storage method not only limits real-time data reading, but also worsens the difficulty of data recovery due to base insertion or deletion.

### SUMMARY

The present disclosure provides a spatially layered DNA storage method for large-scale oligo pools, and proposes a spatially layered DNA data storage method, which integrates spatially layered DNA encoding, base run-length sequence merging, real-time coding base layer forming, and run-length feedback error correction, and can achieve real-time read-out of DNA storage data. Details please find the following description:

A spatially layered DNA storage method for large-scale oligo pools, includes the following steps:
(1) grouping user data into L (20≤L≤500) data layers having a regular length of K (K being a positive integer) bits, performing error correction encoding on each individual data layer respectively to obtain an encoded data layer having a length of N (N>K) bits, and according to a determined mapping rule between bit pairs and bases, transcoding the encoded data layer to obtain a coding base layer;
(2) according to different sequencing read-out modes, sequentially allocating bases in the coding base layer to positions from 1 to L of a DNA sequence, to constitute a payload part of a single-end read DNA sequence having a length of L, and sequentially allocating the coding base layer to symmetrical positions at two ends of the DNA sequence, to obtain a payload part of a paired-end read DNA sequence having a length of 2L;
(3) adding indices and primers to generated DNA data bearing sequences, to obtain large-scale oligonucleotide DNA sequences;
(4) performing sequencing library preparation on each oligo pool obtained by synthesis, and further, performing simultaneously sequencing and readout via a high-throughput sequencing technology for aligning and immobilizing oligonucleotides on a solid-phase carrier;
(5) subjecting oligonucleotide sequences aligned and immobilized on a surface of the solid-phase carrier to real-time optical signal or electrical signal acquisition, signal analysis, incremental base calling, and primer and index identification, and when performing depolymerization detection by using naturally unmodified deoxy-ribonucleoside triphosphate (dNTP), obtaining partial run-length sequences in a base run-length metric form, wherein the base run-length represents a length of a continuous base obtained by recognition when a current nucleotide is used for polymerization; and when performing depolymerization detection by using modified dNTP with a terminator and a fluorescent group, obtaining a presence or absence of a signal by optical detection, i.e., judging a presence or absence of a single base;
(6) according to the identified primers and indices, obtaining multi-copy signals in different base signal forms, clustering the signals, then performing multi-copy merging of the signals, and transforming same to generate consensus run-length sequences;
(7) according to whether there are insertion and deletion errors during sequencing, setting a run-length sequence feedback update mechanism, and in a case where there are no insertion and deletion errors, directly transforming the consensus run-length sequences into coding base layers; and in a case where there is insertion and deletion error propagation, updating the partial run-length sequences by using a feedback result of successful decoding of a previous layer, generating consensus run-length sequences by using multi-copy majority voting, then transforming consensus partial run-length sequences into partial base sequences according to a determined reference base sequence, and sequentially allocating the partial base sequences to determined positions of the coding base layers, and forming individual coding base layers by bases at same positions; and
(8) counting and updating base available ratios of all the coding base layers, performing threshold comparison, outputting the coding base layers that are not successfully decoded, sending same to a decoder for decoding, and recovering original user data layer by layer.

The grouping user data into L data layers having a regular length of K, performing error correction encoding on each individual data layer respectively to obtain an encoded data layer having a length of N(N>K), and according to a determined mapping rule between bit pairs and bases, transcoding the encoded data layer to obtain an coding base layer, has the following specific steps:
(1.1) averagely dividing user data into L groups that correspond to L data layers, wherein a size of each layer of data is K bits;
(1.2) scrambling individual data layers by superposing same-length pseudorandom sequences, and encoding same by using a linear block code (N, K) to obtain encoded data layers having a size of N bits; and traversing all the L data layers, and repeatedly executing above operations, to obtain L encoded data layers; and
(1.3) according to the determined mapping rule between bit pairs and bases, i.e., {00→A, 01→T, 10→G, 11→C}, transcoding the L encoded data layers respectively to obtain L coding base layers.

The according to different sequencing read-out modes, sequentially allocating bases in the coding base layer to positions from 1 to L of a DNA sequence, to constitute a payload part of a single-end read DNA sequence having a length of L, and sequentially allocating the coding base layer to symmetrical positions at two ends of the DNA sequence, to obtain a payload part of a paired-end read DNA sequence having a length of 2L, has the following specific steps:
(2.1) taking out one base in sequence from same positions of the L coding base layers, and allocating same in sequence to positions from 1 to L of a data DNA sequence; and traversing all positions of the coding base layers, and repeatedly executing same steps, to obtain N/2 single-end read DNA sequences having a length of L bases, wherein a base length d of an oligonucleotide sequence constructing a payload part satisfies 20≤d≤500; and
(2.2) taking out two bases respectively from same positions of the L coding base layers, and according to a basic criterion that a first layer of bases is located outside a sequence and a last layer of bases is located inside the sequence, splicing base pairs to same positions at two ends of a symmetrical DNA sequence, respectively, to constitute a payload part of a single paired-end read DNA sequence having a length of 2L bases; and traversing all positions of the coding base layers, and repeatedly executing same steps, to obtain N/4 paired-end read data DNA sequences having a length of 2L bases, wherein a base length d of an oligonucleotide sequence constructing a payload part satisfies 20≤d≤500.

The adding indices and primers to generated DNA data bearing sequences, to obtain large-scale oligonucleotide DNA sequences, is specifically as follows: adding a unique index having a base length of n to two ends of each DNA sequence, respectively, wherein different oligo pools share a same group of indices, further adding a fixed-length primer to a single end/paired ends of DNA sequences, to construct complete oligo pools, and performing high-throughput synthesis.

The performing sequencing library preparation on each oligo pool obtained by synthesis, and further, performing read-out by sequencing by means of a high-throughput sequencing technology for aligning and immobilizing oligonucleotides on a solid-phase carrier, is specifically as follows:

(3.1) corresponding to paired-end symmetric mapping construction of oligonucleotide sequences of the coding base layers, by using a standard library preparation method, performing polymerase chain reaction amplification on the synthetic oligo pools by using forward and reverse primer pairs, respectively, taking out a small number of samples for adding sequencing adapters, and constructing a sequencing library which is loaded to a sequencer for sequencing;
(3.2) corresponding to paired-end symmetric mapping construction of oligonucleotide sequences of the coding base layers, by using a bidirectional library preparation method, performing polymerase chain reaction amplification on the oligo pools by using paired forward and reverse overhanging primers, respectively, to obtain two sequencing libraries which are then mixed, and taking out a small number of samples for single-template amplification on a solid-phase carrier, such as a magnetic bead, including an overhanging primer complementary sequence, to complete preparation of sequencing libraries which are loaded to a sequencer for sequencing; and
(3.3) corresponding to single-end asymmetric mapping construction of oligonucleotide sequences of the coding base layers, by using a single-end library preparation method, performing polymerase chain reaction amplification on the synthetic oligo pools only by using a forward primer by using fixed amplification in one direction, taking out a small number of samples for single-template amplification on a solid-phase carrier, such as a magnetic bead, including an overhanging primer complementary sequence, to complete preparation of sequencing libraries which are loaded to a sequencer for sequencing.

The subjecting oligonucleotide sequences aligned and immobilized on a surface of the solid-phase carrier to real-time optical signal or electrical signal acquisition, signal analysis, incremental base calling, and primer and index identification, to obtain partial run-length sequences in a base run-length metric form, has the following specific steps:
(4.1) during incremental base calling, recording base calling results in real time, and by using a base run-length metric criterion, according to a determined reference base sequence, mapping partial base sequences obtained by base calling to partial run-length sequences; and
(4.2) performing primer identification on the identified partial run-length sequences by using a run space distance metric, determining a boundary thereof, starting from a primer recognition boundary, intercepting the partial run-length sequences, demapping same into base sequences having a same length as that of an index part, pre-processing the index part, performing validity checking or error correction by using a check matrix to retain an information part of valid partial run-length sequences, and labeling corresponding primers, indices and relative start offset from a reference run-length sequence.

The according to the identified primers and indices, obtaining multi-copy signals in different base signal forms, clustering the signals, then performing multi-copy merging of the signals, and transforming same to generate consensus run-length sequences, has the following specific steps:
(5.1) counting one by one a base run-length metric value corresponding to each run-length sequence of the multi-copy signals;
(5.2) executing majority voting, and outputting a most frequent base run-length metric; and
(5.3) transforming into a base sequence according to the base run-length metric value, and outputting layer by layer a base sequence after multi-copy merging.

The according to whether there are insertion and deletion errors during sequencing, setting a run-length sequence feedback update mechanism, and in a case where there are no insertion and deletion errors, directly transforming the consensus run-length sequences into coding base layers; and in a case where there is insertion and deletion error propagation, updating the partial run-length sequences by using a feedback result of successful decoding of a previous layer, generating consensus run-length sequences by using multi-copy majority voting, then transforming consensus partial run-length sequences into partial base sequences according to a determined reference base sequence, and sequentially allocating the partial base sequences to determined positions of the coding base layers, and forming individual coding base layers by bases at same positions, has the following specific steps:
(6.1) updating multiple copies of partial run-length sequences having same primers and indices by using the feedback result of successful decoding of the previous layer, then performing majority voting position by position, generating consensus partial run-length sequences, demapping the consensus partial run-length sequences into partial base sequences according to a determined reference base sequence, and sequentially placing the partial base sequences at determined positions of the L coding base layers according to the identified primers and indices, wherein each coding base layer includes N/2 unique sites;
(6.2) counting and updating a base available ratio of each individual coding base layer, if the ratio reaches a preset threshold of successful decoding, outputting coding base layers that have not been successfully decoded, further, transforming the output coding base layers into bits, performing error correction by using the linear block code, and recovering and obtaining user data corresponding to current data layers; and
(6.3) generating ideal partial base sequences by using coding base layers that are successfully decoded previously, transforming same according to the determined reference base sequence into ideal partial run-length sequences, generating total N/2 partial run-length sequences, and feeding back the partial run-length sequences generated again to steps (5.1) to (5.3) to re-execute majority voting.

The counting and updating base available ratios of all the coding base layers, performing threshold comparison, outputting the coding base layers that are not successfully decoded, sending same to a decoder for decoding, and recovering original user data layer by layer, has the following specific steps:
(7.1) transforming the generated coding base layers after merging into bits, and decoding same by using the linear block code for error correction; and
(7.2) sequentially executing merging and decoding of each layer of data until all layers of data are completely read out, to achieve real-time DNA storage readout through simultaneous sequencing and decoding.

The scrambling individual data layers by superposing same-length pseudorandom sequences, and encoding same by using a linear block code (N, K), to obtain encoded data layers having a size of N bits; and traversing all the L data layers, and repeatedly executing same operations, to obtain L encoded data layers, has the following specific steps:
(8.1) encoding data by a linear block code using a product code, and dividing each layer of data into P data blocks having a same size;
(8.2) executing an encoding process of a first component code in product code encoding on P data blocks in each layer, adding a check data block, and generating M data blocks, to constitute codeword blocks of the first component code of a single layer;
(8.3) executing an encoding process of a second component code in product code encoding, encoding each data block in each layer into the codeword of the second component code by using a generation matrix, and generating total M codewords of the second component code in each layer; and
(8.4) traversing all the L data layers, and repeatedly executing a product code encoding process, to finally obtain L encoded data layers.

The adding indices and primers to generated DNA data bearing sequences, to obtain large-scale oligonucleotide DNA sequences, being specifically as follows: adding a unique index having a base length of n to two ends of each DNA sequence, respectively, wherein different oligo pools share a same group of indices, further adding a fixed-length primer to a single end/paired ends of DNA sequences, to construct complete oligo pools, and performing high-throughput synthesis, has the following specific steps:
(9.1) traversing all natural numbers within a range of [0, 2^{k}], representing same by using bit vectors having a length of k bits, encoding each bit vector by using a binary short block error correction code (n, k), and randomly interleaving obtained codewords, wherein, k≥┌log₂\hat{N}┐ and is a positive integer, \hat{N} represents the number of base sequences included in the oligo pools, and ┌g┐ represents rounding up to an integer;
(9.2) transcoding encoded codeword sequences according to a determined mapping rule to obtain index sequences having a base length of n/2, counting sequence homopolymer length distribution, according to a minimum homopolymer length criterion, preferentially selecting a base sequence having a smaller homopolymer length, and for single-end read DNA sequences, screening N/2 sequences as valid indices; and for paired-end read DNA sequences, screening N/4 sequences as valid indices; and
(9.3) adding the index base sequences to forward ends of data DNA sequences, then randomly interleaving index DNA sequences at a base level, and adding same to the other ends of the data DNA sequences, to generate DNA sequences where index identification can be performed at two ends.

The performing primer identification on the identified partial run-length sequences by using a run space distance metric, determining a boundary thereof, starting from a primer recognition boundary, intercepting the partial run-length sequences, demapping same into base sequences having a same length as that of an index part, pre-processing the index part, performing validity checking or error correction by using a check matrix to retain an information part of valid partial run-length sequences, and labeling corresponding primers, indices and relative start offset from a reference run-length sequence, has the following specific steps:
(10.1) by using a known reference base sequence, constructing reference run-length sequences of all forward and reverse primers used in the oligo pools, comparing partial run-length sequences identified after accumulation during real-time base calling with the reference run-length sequences to obtain a spatial distance, and determining primer classifications to which corresponding partial sequences belong;
(10.2) searching for a primer having a minimum run spatial distance according to a comparison result, comparing the run spatial distance thereof with a preset threshold, if the preset threshold is satisfied, considering that the primer is valid, and retaining a corresponding sequence and labeling a most possible primer; otherwise, discarding the corresponding run-length sequence;
(10.3) demapping index part of base sequences to bit sequences, and deinterleaving same to obtain codewords corresponding to the indices; and
(10.4) performing validity checking by using a check matrix, if checking is correct and the indices are legal, considering that the indices are valid, and retaining run-length sequences of corresponding information part; and performing error correction on index part by using a short block error correction code, recovering original indices, checking index legality, and retaining run-length sequences of corresponding information part.

The updating multiple copies of partial run-length sequences having same primers and indices by using the feedback result of successful decoding of the previous layer, then performing majority voting position by position, generating consensus partial run-length sequences, demapping the consensus partial run-length sequences into partial base sequences according to a determined reference base sequence, and sequentially placing the partial base sequences at determined positions of the L coding base layers according to the identified primers and indices, wherein each coding base layer includes N/2 unique sites, has the following specific steps:
(11.1) for multiple copies of partial run-length sequences having same primers and indices, aligning the partial run-length sequences according to a start offset relative to a reference run-length sequence, comparing position-by-position values of original run-length sequences with updated values by using the feedback result of successful decoding of the previous layer, and if original values are greater than the updated values, retaining the original values; otherwise, refreshing by using the updated values;
(11.2) performing position-by-position majority voting on the partial run-length sequences having multiple copies, to obtain consensus partial run-length sequences; and if a voting result at a certain position is not unique, taking a base run-length corresponding to frequency suboptimality as a consensus voting result at the position; and
(11.3) demapping the consensus partial run-length sequences into partial base sequences according to a determined reference base sequence, and placing the partial base sequences at determined positions of the coding base layers according to the determined primers and indices.

The technical solution provided by the present disclosure has the following beneficial effects:

The present disclosure proposes a data layered coding method based on spatial layering, which can store data layer by layer in a large number of oligonucleotides, makes full use of arrangement characteristics of oligo pools, and matches sequencing processes, thereby realizing real-time sequencing and data decoding and readout. The present disclosure proposes a sequence consensus method based on run-length sequence merging and a run-length sequence iteration feedback mechanism, and values of corresponding positions of run-length sequences are updated by using a feedback result of successful decoding of a previous layer, so that the error rate in data layers can be reduced, and cross-layer error propagation is eliminated; and error correction is performed in combination with linear block code decoding, thereby achieving real-time layer-wise readout of user data.

### BRIEF DESCRIPTION OF THE DRAWINGS

FIG. 1 shows a system chart of a spatial layered DNA storage method for large-scale oligo pools provided by the present disclosure;

FIG. 2 shows a flowchart of a spatial layered encoding method provided by the present disclosure;

FIG. 3 shows a schematic diagram of a paired-end library preparation principle provided by the present disclosure;

FIG. 4 shows a flowchart of a stream layered real-time readout method provided by the present disclosure;

FIG. 5 shows a flowchart of majority voting of run-length sequences provided by the present disclosure;

FIG. 6 shows a flowchart of feedback update of run-length sequences based on partial successful decoding results provided by the present disclosure;

FIG. 7 shows a flowchart of constructing coding base layers based on partial run-length sequences provided by the present disclosure;

FIG. 8 shows a flowchart of layered DNA data storage of DNA spatially layered encoding provided by the present disclosure;

FIG. 9 shows a flowchart of decomposing a movie file into 28 layers in time order provided by the present disclosure;

FIG. 10 shows a schematic diagram of intra-layer data block encoding using two-dimensional product encoding provided by the present disclosure;

FIG. 11 shows a schematic diagram of two-dimensional product codes constructed based on RS codes and LDPC codes provided by the present disclosure;

FIG. 12 shows a schematic structural diagram of oligonucleotide molecules provided by the present disclosure;

FIG. 13 shows a schematic diagram of an error correction principle of intra-layer two-dimensional product codes provided by the present disclosure;

FIG. 14 shows a diagram of cumulative probability distribution of primers and indices based on a base run-length metric criteria for determined sequences provided by the present disclosure;

FIG. 15 shows a comparison diagram of error rate performance at a signal level before and after majority voting of multi-copy signals provided by the present disclosure;

FIG. 16 is a performance comparison diagram of majority voting of multi-copy signals and feedback update provided by the present disclosure; and

FIG. 17 shows error-free recovery of data under different sequencing coverage provided by the present disclosure.

### DESCRIPTION OF THE EMBODIMENTS

To make the objectives, technical solutions and advantages of the present disclosure clearer, the implementations of the present disclosure will be further described in detail below.

The implementations of the present disclosure are described in detail below in combination with drawings.

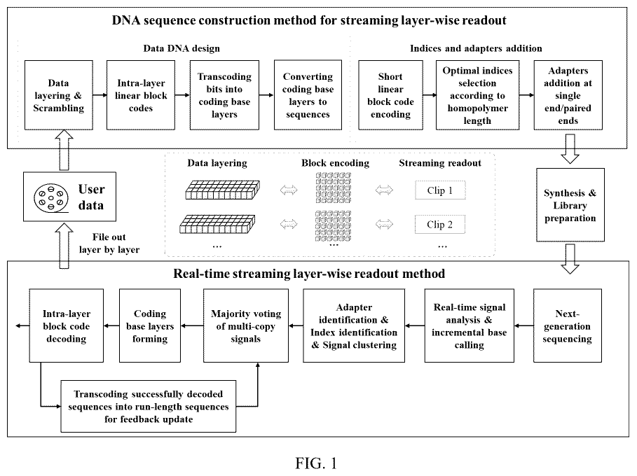

This implementation introduces in detail a spatially layered DNA storage method for large-scale oligo pools proposed by the present disclosure, FIG. 1 shows a complete implementation process, and the method specifically includes the following steps:

(1) grouping user data into L (20≤L≤500) data layers having a regular length of K (K being a positive integer) bits, performing error correction encoding on each individual data layer respectively to obtain an encoded data layer having a length of N (N>K) bits, and according to a determined mapping rule between bit pairs and bases, transcoding the encoded data layer to obtain a coding base layer;
(2) according to different sequencing read-out modes, sequentially allocating bases in the coding base layer to positions from 1 to L of a DNA sequence, to constitute a payload part of a single-end read DNA sequence having a length of L, and sequentially allocating the coding base layer to symmetrical positions at two ends of the DNA sequence, to obtain a payload part of a paired-end read DNA sequence having a length of 2L;
(3) adding indices and primers to generated DNA data bearing sequences, to obtain large-scale oligonucleotide DNA sequences;
(4) performing sequencing library preparation on each oligo pool obtained by synthesis, and further, performing simultaneously sequencing and readout via the high-throughput sequencing technology for aligning and immobilizing oligonucleotides on a solid-phase carrier;
(5) subjecting oligonucleotide sequences aligned and immobilized on a surface of the solid-phase carrier to real-time optical signal or electrical signal acquisition, signal analysis, incremental base calling, and primer and index identification, and when performing depolymerization detection by using naturally unmodified deoxy-ribonucleoside triphosphate (dNTP), obtaining partial run-length sequences in a base run-length metric form, wherein the base run-length represents a length of a continuous base obtained by recognition when a current nucleotide is used for polymerization; and when performing depolymerization detection by using modified dNTP with a terminator and a fluorescent group, obtaining a presence or absence of a signal by optical detection, i.e., judging a presence or absence of a single base;
(6) according to the identified primers and indices, obtaining multi-copy signals in different base signal forms, clustering the signals, then performing multi-copy merging of the signals, and transforming same to generate consensus run-length sequences;
(7) according to whether there are insertion and deletion errors during sequencing, setting a run-length sequence feedback update mechanism, and in a case where there are no insertion and deletion errors, directly transforming the consensus run-length sequences into coding base layers; and in a case where there is insertion and deletion error propagation, updating the partial run-length sequences by using a feedback result of successful decoding of a previous layer, generating consensus run-length sequences by using multi-copy majority voting, then transforming consensus partial run-length sequences into partial base sequences according to a determined reference base sequence, and sequentially allocating the partial base sequences to determined positions of the coding base layers, and forming individual coding base layers by bases at same positions; and
(8) counting and updating base available ratios of all the coding base layers, by threshold comparison, outputting the coding base layers that are not successfully decoded, sending same to a decoder for decoding, and recovering original user data layer by layer.

The step (1) grouping user data into L (20≤L≤500) data layers having a regular length of K (K being a positive integer) bits, performing error correction encoding on each individual data layer respectively to obtain an encoded data layer having a length of N(N>K) bits, and according to a determined mapping rule between bit pairs and bases, transcoding the encoded data layer to obtain an coding base layer, has the following specific operations:
(1.1) averagely dividing user data into L groups that correspond to L data layers, wherein a size of each layer of data is K bits;
(1.2) scrambling individual data layers by superposing same-length pseudorandom sequences, and encoding same by using a linear block code (N, K), to obtain encoded data layers having a size of N bits; and traversing all the L data layers, and repeatedly executing same operations, to obtain L encoded data layers; and
(1.3) according to the determined mapping rule between bit pairs and bases, i.e., {00→A, 01→T, 10→G, 11→C}, transcoding the L encoded data layers respectively to obtain L coding base layers.

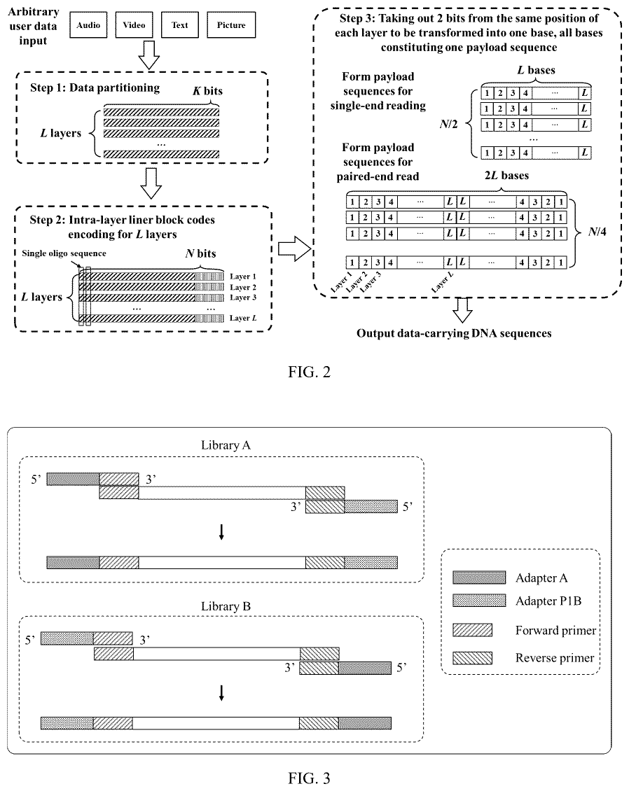

The step (2) according to different sequencing read-out modes, sequentially allocating bases in the coding base layer to positions from 1 to L of a DNA sequence, to constitute a payload part of a single-end read DNA sequence having a length of L, and sequentially allocating the coding base layer to symmetrical positions at two ends of the DNA sequence, to obtain a payload part of a paired-end read DNA sequence having a length of 2L, FIG. 2 shows an implementation process, has the following specific operations:
(2.1) taking out one base in sequence from same positions of the L coding base layers, and allocating same in sequence to positions from 1 to L of a data DNA sequence; and traversing all positions of the coding base layers, and repeatedly executing same steps, to obtain N/2 single-end read DNA sequences having a length of L bases, wherein a base length d of an oligonucleotide sequence constructing a payload part satisfies 20≤d≤500; and
(2.2) taking out two bases respectively from same positions of the L coding base layers, and according to a basic criterion that a first layer of bases is located outside a sequence and a last layer of bases is located inside the sequence, splicing base pairs to same positions at two ends of a symmetrical DNA sequence, respectively, to constitute a payload part of a single paired-end read DNA sequence having a length of 2L bases; and traversing all positions of the coding base layers, and repeatedly executing same steps, to obtain N/4 paired-end read data DNA sequences having a length of 2L bases, wherein a base length d of an oligonucleotide sequence constructing a payload part satisfies 20≤d≤500.

The step (3) adding indices and primers to generated DNA data bearing sequences, to obtain large-scale oligonucleotide DNA sequences, is specifically as follows: adding a unique index having a base length of n to two ends of each DNA sequence, respectively, wherein different oligo pools share a same group of indices, further adding a fixed-length primer to a single end/paired ends of DNA sequences, to construct complete oligo pools, and performing high-throughput synthesis.

The step (4) performing sequencing library preparation on each oligo pool obtained by synthesis, and further, performing read-out by sequencing by means of a high-throughput sequencing technology for aligning and immobilizing oligonucleotides on a solid-phase carrier, has the following specific operations:
(3.1) corresponding to paired-end symmetric mapping construction of oligonucleotide sequences of the coding base layers, by using a standard library preparation method, performing polymerase chain reaction amplification on the synthetic oligo pools by using forward and reverse primer pairs, respectively, taking out a small number of samples for adding sequencing adapters, and constructing a sequencing library which is loaded to a sequencer for sequencing;
(3.2) corresponding to paired-end symmetric mapping construction of oligonucleotide sequences of the coding base layers, by using a bidirectional library preparation method, performing polymerase chain reaction amplification on the oligo pools by using paired forward and reverse overhanging primers, respectively, to obtain two sequencing libraries which are then mixed, and taking out a small number of samples for single-template amplification on a solid-phase carrier, such as a magnetic bead, including an overhanging primer complementary sequence, to complete preparation of sequencing libraries which are loaded to a sequencer for sequencing; and
(3.3) corresponding to single-end asymmetric mapping construction of oligonucleotide sequences of the coding base layers, by using a single-end library preparation method, performing polymerase chain reaction amplification on the synthetic oligo pools only by using a forward primer by using fixed amplification in one direction, taking out a small number of samples for single-template amplification on a solid-phase carrier, such as a magnetic bead, including an overhanging primer complementary sequence, to complete preparation of sequencing libraries which are loaded to a sequencer for sequencing.

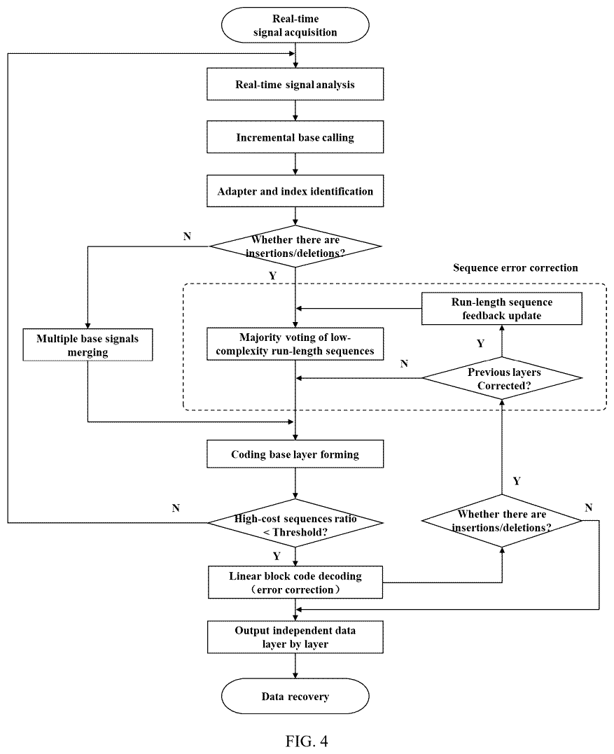

The step (5) subjecting oligonucleotide sequences aligned and immobilized on a surface of the solid-phase carrier to real-time optical signal or electrical signal acquisition, signal analysis, incremental base calling, and primer and index identification, and when performing depolymerization detection by using naturally unmodified deoxy-ribonucleoside triphosphate (dNTP), obtaining partial run-length sequences in a base run-length metric form, wherein the base run-length represents a length of a continuous base obtained by recognition when a current nucleotide is used for polymerization; and when performing depolymerization detection by using modified dNTP with a terminator and a fluorescent group, obtaining a presence or absence of a signal by optical detection, i.e., judging a presence or absence of a single base, FIG. 4 shows a specific recovery process thereof, has the following specific operations:
(4.1) during incremental base calling, recording base calling results in real time, and by using a base run-length metric criterion, according to a determined reference base sequence, mapping partial base sequences obtained by base calling to partial run-length sequences; and
(4.2) performing primer identification on the identified partial run-length sequences by using a run space distance metric, determining a boundary thereof, starting from a primer recognition boundary, intercepting the partial run-length sequences, demapping same into base sequences having a same length as that of an index part, pre-processing the index part, performing validity checking or error correction by using a check matrix to retain an information part of valid partial run-length sequences, and labeling corresponding primers, labels and relative start offset from a reference run-length sequence.

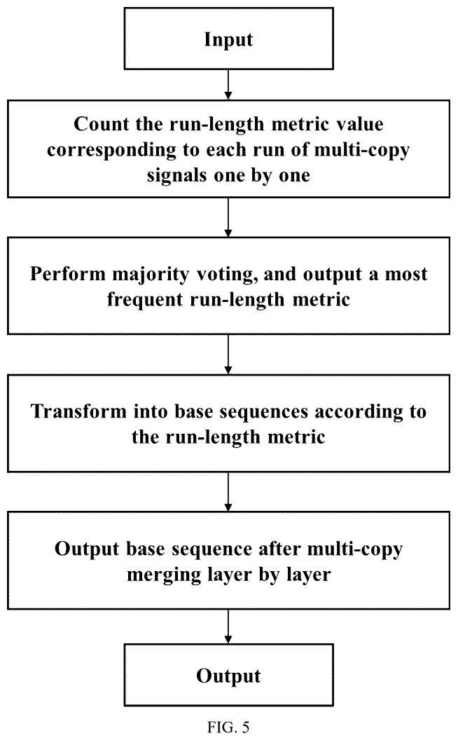

The step (6) according to the identified primers and indices, obtaining multi-copy signals in different base signal forms, clustering the signals, then performing multi-copy merging of the signals, and transforming same to generate consensus run-length sequences, FIG. 5 shows a majority voting process, has the following specific operations:
(5.1) counting one by one a base run-length metric value corresponding to each run-length sequence of the multi-replica signals;
(5.2) executing majority voting, and outputting a most frequent base run-length metric; and
(5.3) transforming into a base sequence according to the base run-length metric value, and outputting layer by layer a base sequence after multi-copy merging.

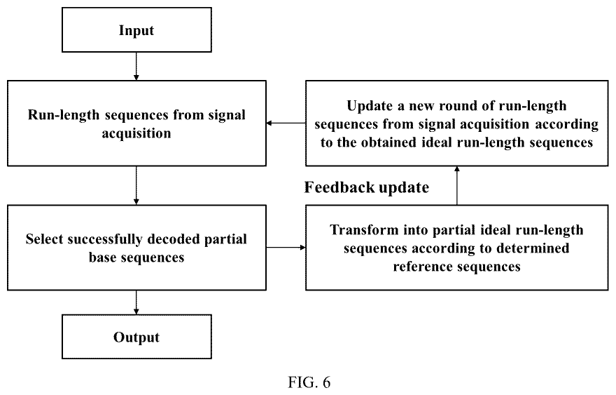

The step (7) according to whether there are insertion and deletion errors during sequencing, setting a run-length sequence feedback update mechanism, and in a case where there are no insertion and deletion errors, directly transforming the consensus run-length sequences into coding base layers; and in a case where there is insertion and deletion error propagation, updating the partial run-length sequences by using a feedback result of successful decoding of a previous layer, generating consensus run-length sequences by using multi-copy majority voting, then transforming consensus partial run-length sequences into partial base sequences according to a determined reference base sequence, and sequentially allocating the partial base sequences to determined positions of the coding base layers, and forming individual coding base layers by bases at same positions, FIG. 6 shows a process of feedback update of run-length sequences based on partial correct decoding results, has the following specific operations:
(6.1) updating multiple copies of partial run-length sequences having same primers and indices by using the feedback result of successful decoding of the previous layer, then performing majority voting position by position, generating consensus partial run-length sequences, demapping the consensus partial run-length sequences into partial base sequences according to a determined reference base sequence, and sequentially placing the partial base sequences at determined positions of the L coding base layers according to the identified primers and indices, wherein each coding base layer includes N/2 unique sites;
(6.2) counting and updating a base available ratio of each individual coding base layer, if the ratio reaches a preset threshold of successful decoding, outputting coding base layers that have not been successfully decoded, further, transforming the output coding base layers into bits, performing error correction by using the linear block code, and recovering and obtaining user data corresponding to current data layers; and
(6.3) generating ideal partial base sequences by using coding base layers that are successfully decoded previously, transforming same according to the determined reference base sequence into ideal partial run-length sequences, generating total N/2 partial run sequences, and feeding back the partial run-length sequences generated again to steps (5.1) to (5.3) to re-execute majority voting.

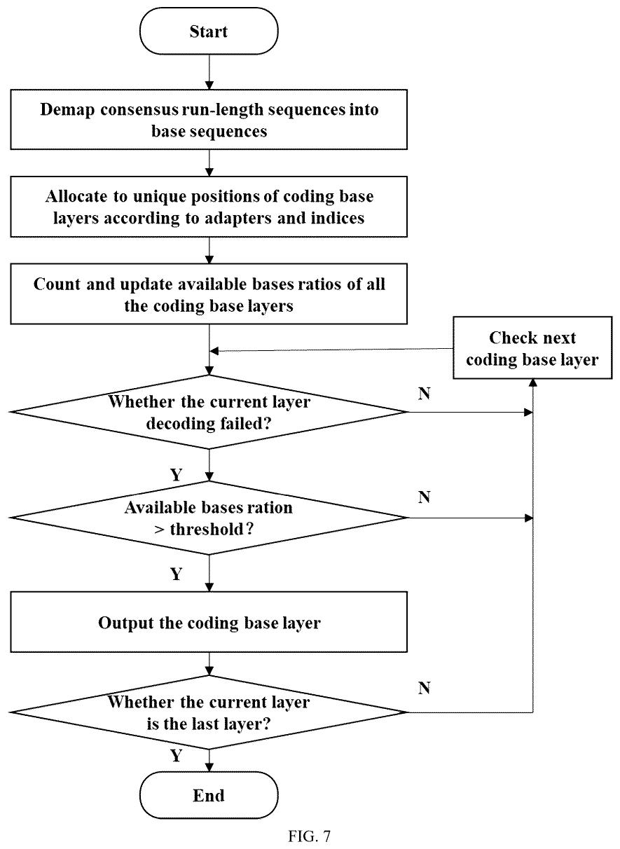

The step (8) counting and updating base available ratios of all the coding base layers, by threshold comparison, outputting the coding base layers that are not successfully decoded, sending same to a decoder for decoding, and recovering original user data layer by layer, FIG. 7 shows an encoded base generation process, has the following specific operations:
(7.1) transforming the generated coding base layers after merging into bits, and decoding same by using the linear block code for error correction; and
(7.2) sequentially executing merging and decoding of each layer of data until all layers of data are completely read out, to achieve real-time DNA storage readout through simultaneous sequencing and decoding.

The step (1.2) scrambling individual data layers by superposing same-length pseudorandom sequences, and encoding same by using a linear block code (N, K), to obtain encoded data layers having a size of N bits; and traversing all the L data layers, and repeatedly executing same operations, to obtain L encoded data layers, has the following specific operations:
(8.1) encoding data by a linear block code using a product code, and dividing each layer of data into P data blocks having a same size;
(8.2) executing an encoding process of a first component code in product code encoding on P data blocks in each layer, adding a check data block, and generating M data blocks, to constitute codeword blocks of the first component code of a single layer;
(8.3) executing an encoding process of a second component code in product code encoding, encoding each data block in each layer into the codeword of the second component code by using a generation matrix, and generating total M codewords of the second component code in each layer; and
(8.4) traversing all the L data layers, and repeatedly executing a product code encoding process, to finally obtain L encoded data layers.

The step of adding indices and primers to generated DNA data bearing sequences, to obtain large-scale oligonucleotide DNA sequences, being specifically as follows: adding a unique index having a base length of n to two ends of each DNA sequence, respectively, wherein different oligo pools share a same group of indices, further adding a fixed-length primer to a single end/paired ends of DNA sequences, to construct complete oligo pools, and performing high-throughput synthesis, has the following specific operations:
(9.1) traversing all natural numbers within a range of [0, 2^{k}], representing same by using bit vectors having a length of k bits, encoding each bit vector by using a binary short block error correction code (n, k), and randomly interleaving obtained codewords, wherein, k≥┌log₂\hat{N}┐ and is a positive integer, \hat{N} represents the number of base sequences included in the oligo pools, and ┌g┐ represents rounding up to an integer;
(9.2) transcoding encoded codeword sequences according to a determined mapping rule to obtain index sequences having a base length of n/2, counting sequence homopolymer length distribution, according to a minimum homopolymer length criterion, preferentially selecting a base sequence having a smaller homopolymer length, and for single-end read DNA sequences, screening N/2 sequences as valid indices; and for paired-end read DNA sequences, screening N/4 sequences as valid indices; and
(9.3) adding the index base sequences to forward ends of data DNA sequences, then randomly interleaving index DNA sequences at a base level, and adding same to the other ends of the data DNA sequences, to generate DNA sequences where index identification n can be performed at two ends.

The step (4.2) performing primer identification on the identified partial run-length sequences by using a run space distance metric, determining a boundary thereof, starting from a primer recognition boundary, intercepting the partial run-length sequences, demapping same into base sequences having a same length as that of an index part, pre-processing the index part, performing validity checking or error correction by using a check matrix to retain an information part of valid partial run-length sequences, and labeling corresponding primers, labels and relative start offset from a reference run-length sequence, has the following specific operations:
(10.1) by using a known reference base sequence, constructing reference run-length sequences of all forward and reverse primers used in the oligo pools, comparing partial run-length sequences identified after accumulation during real-time base calling with the reference run-length sequences to obtain a spatial distance, and determining primer classifications to which corresponding partial sequences belong;
(10.2) searching for a primer having a minimum run spatial distance according to a comparison result, comparing the run spatial distance thereof with a preset threshold, if the preset threshold is satisfied, considering that the primer is valid, and retaining a corresponding sequence and labeling a most possible primer; otherwise, discarding the corresponding run-length sequence;
(10.3) demapping index part of base sequences to bit sequences, and deinterleaving same to obtain codewords corresponding to the indices; and
(10.4) performing validity checking by using a check matrix, if checking is correct and the indices are legal, considering that the indices are valid, and retaining run-length sequences of corresponding information part; and performing error correction on index part by using a short block error correction code, recovering original indices, checking index legality, and retaining run-length sequences of corresponding information part.

The step (6.1) updating multiple copies of partial run-length sequences having same primers and indices by using the feedback result of successful decoding of the previous layer, then performing majority voting position by position, generating consensus partial run-length sequences, demapping the consensus partial run-length sequences into partial base sequences according to a determined reference base sequence, and sequentially placing the partial base sequences at determined positions of the L coding base layers according to the identified primers and indices, wherein each coding base layer includes N/2 unique sites, has the following specific operations:
(11.1) for multiple copies of partial run-length sequences having same primers and indices, aligning the partial run-length sequences according to a start offset relative to a reference run-length sequence, comparing position-by-position values of original run-length sequences with updated values by using the feedback result of successful decoding of the previous layer, and if original values are greater than the updated values, retaining the original values; otherwise, refreshing by using the updated values;
(11.2) performing position-by-position majority voting on the partial run-length sequences having multiple copies, to obtain consensus partial run-length sequences; and if a voting result at a certain position is not unique, taking a base run-length corresponding to frequency suboptimality as a consensus voting result at the position; and
(11.3) demapping the consensus partial run-length sequences into partial base sequences according to a determined reference base sequence, and placing the partial base sequences at determined positions of the coding base layers according to the determined primers and indices.

### SPECIFIC EMBODIMENTS

Specific embodiments will be given below in conjunction with the drawings to describe in detail the feasibility of a spatially layered DNA storage method for large-scale oligo pools provided by the present disclosure.

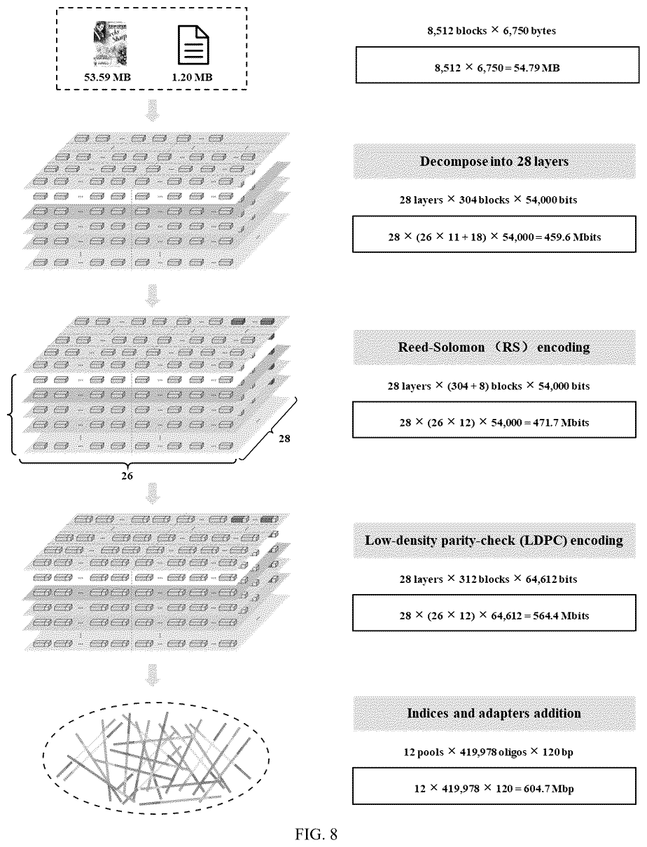

Stored files in this embodiment are a film file having a size of 53.59 MB, and a text file herein having a size of 1.20 MB. The files are encoded into about 10.08 million oligonucleotide sequences by using the spatially layered codes to form large-scale oligo pools, and in conjunction with the Ion Torrent sequencing technology, a real-time readout rate of about 500 Kbit/s is achieved. Processes of encoding, sequencing, error correction and real-time readout of the film file specifically include the following steps:
(1) splitting user data having a total size of 54.79 MB in time order into 28 data layers having a regular structure, performing two-dimensional product code error correction encoding on each individual data layer respectively to obtain an encoded data layer, transcoding the encoded data layer according to a mapping rule between bit pairs and bases to obtain a coding base layer, and then sequentially allocating bases in the coding base layer to symmetric positions at two ends of a DNA sequence; and grouping generated data DNA sequences and adding address indices and primers, wherein a specific spatially layered encoding process is as shown in FIG. 8;
(2) performing multiple rounds of polymerase chain reaction amplification on oligo pools obtained by synthesis, taking out a small number of samples from amplified samples for bidirectional library preparation, and further, performing simultaneous sequencing and readout via the semiconductor rapid sequencing technology; and
(3) subjecting oligonucleotide sequences aligned and immobilized on a surface of a solid-phase carrier to real-time electrical signal acquisition, signal analysis, incremental base calling, and primer and index identification, to obtain partial run-length sequences in a base run-length metric form, according to identified primers and indices, updating the partial run-length sequences by using a feedback result of successful decoding of a previous layer, generating consensus run-length sequences by using multi-copy majority voting, then transforming consensus partial run-length sequences into partial base sequences according to a determined reference base sequence, sequentially allocating the partial base sequences to determined positions of the coding base layers, forming individual coding base layers by bases at same positions, then counting and updating base available ratios of all the coding base layers, by threshold comparison, outputting the coding base layers that are not successfully decoded, sending same to a decoder for decoding, and recovering original user data layer by layer, thereby finally achieving a real-time read-out rate of about 500 Kbit/s.

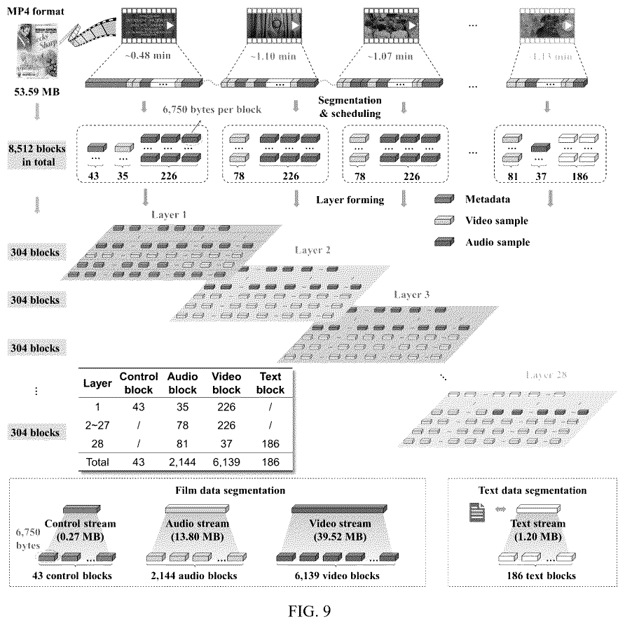

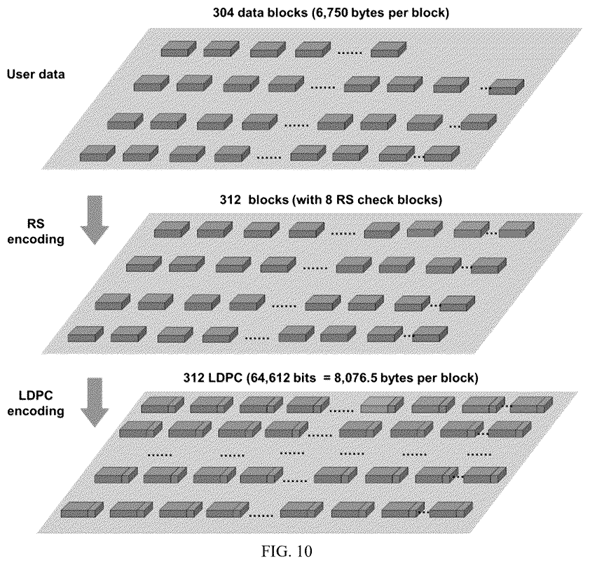

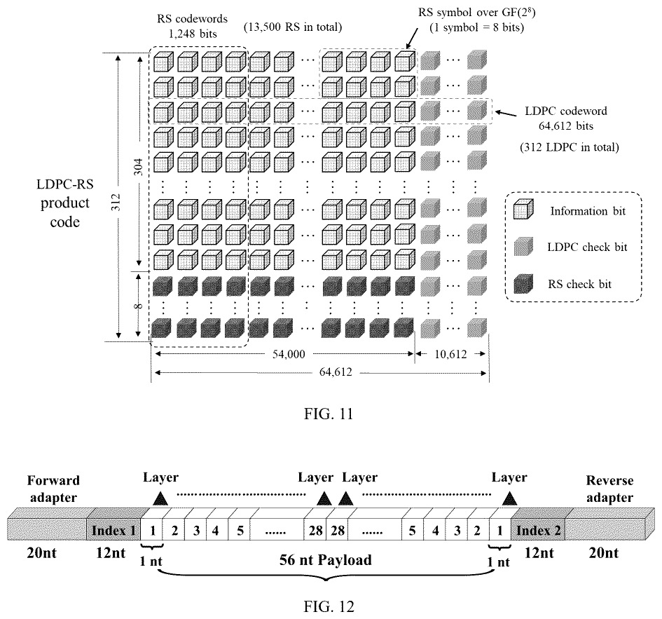

Specific steps of allocating the film file to 28 layers by using spatially layered encoding in step (1) are as shown in FIG. 9, and are specifically as follows:
(1.1) scheduling the film file, and splitting same into 28 groups in time order that correspond to 28 data layers, wherein a size of each layer of data is 2,052,000 bytes;
(1.2) sequentially dividing data corresponding to individual data layers into 304 data blocks each having a size of 6,750 bytes, transforming same into bit sequences of 54,000 bits, and scrambling each bit sequence by superposing same-length pseudorandom sequences, so that 8,512 data blocks in all the 28 layers are basic units that constitute spatially layered encoding;
(1.3) encoding a single data layer by using a two-dimensional product code, wherein the specific processing process thereof is as shown in FIG. 10, firstly, RS encoding is performed, 8 parity blocks are generated at each layer, 4 bits are extracted from each block, 304 blocks form a total of 152 symbols in a Galois Field GF(2⁸), and 4 parity symbols (32 bits) are generated by using RS (156, 152) shortened by RS (255, 251), so that a total of 312 data blocks of 54,000 bits are constructed; then low-density parity-check (LDPC) codes are encoded, in a single layer, a generation matrix is used to encode each data block into one LDPC codeword having an encoded length of 64,612 bits, and a total of 312 encoding data blocks (LDPC codewords) are obtained in each layer, and detailed parameters of RS codes and LDPC codes are as shown in FIG. 11;
(1.4) traversing all the 28 data layers, and repeatedly executing same operations in steps (1.2) and (1.3), to obtain 28 encoded data layers;
(1.5) for the same encoded data layer, dividing 312 blocks in each layer into 12 groups, each including 26 blocks (LDPC codewords), in all the 28 layers, selecting two blocks from each layer in the same group, extracting 56 blocks, then extracting 2 bits from one block every time, transcoding same into two bases according to a determined mapping rule {00→A, 01→T, 10→G, 11→C}, then placing same at symmetrical positions of a single oligonucleotide (payload), and forming 32,306 oligonucleotides, wherein the specific process of mapping the coding base layers into oligonucleotide sequences is as shown in FIG. 12;
(1.6) combining the 26 blocks of all the 28 layers to generate 419,978 oligonucleotide sequences having a length of 56 nucleotides;
(1,7) dividing the generated data DNA sequences into 12 groups on average, corresponding to 12 oligonucleotide pools, so that 5,039,736 oligonucleotide sequences are constructed in total;
(1.8) representing each digit in a range [0, 2¹⁹⁻¹] by using bit vectors having a length of 19 bits; encoding each bit vector by using a shortened Hamming code (formed by shortening a Hamming code with n=31 and k=26) to obtain a codeword having a length of 24 bits, and further randomly interleaving the obtained codeword;
(1.9) transcoding an index codeword into a DNA sequence having a length of 12 nucleotides, screening out all sequences having a homopolymer length less than 4, adding index DNA sequences to one end of a data DNA sequence, randomly interleaving the index DNA sequences at base level, and adding same to the other end of the data DNA sequence, to generate a DNA sequence where indices can be identified at two ends; and
(1.10) designing 12 pairs of primers having a length of 20 nucleotides, wherein in the same synthesis pool, 419,978 oligonucleotide chains of each group share the same pair of primers, 5,039,736 oligonucleotide sequences having a length of 120 nucleotides are constructed by using the total of 12 pairs of primers, and the structure thereof is as shown in FIG. 12.

Specific steps of amplification and sequencing in step (2) are as follows:
(2.1) performing multiple rounds of polymerase chain reaction amplification on each oligo pool by using paired forward and reverse overhanging primers, wherein one pair of overhanging primers extend connectively from adapter A near a proximal end of the oligonucleotide sequence, the other pair start from adapter P1 near a distal end of the oligonucleotide sequence, and exchange the order of the overhanging adapters A and P1;
(2.2) according to the described bidirectional library preparation method, generating a total of 24 libraries by 12 oligo pools, then loading the mixed libraries into an automatic library preparation system for single-chip run, and automatically performing template preparation and chip loading according to manufacturer's instructions; and
(2.3) after completion of the template preparation, loading the prepared sequencing chips into the Ion Torrent sequencer for sequencing, wherein the sequencer is only configured to collect signals, to avoid any subsequent analysis in the sequencer and sequencing procedure.

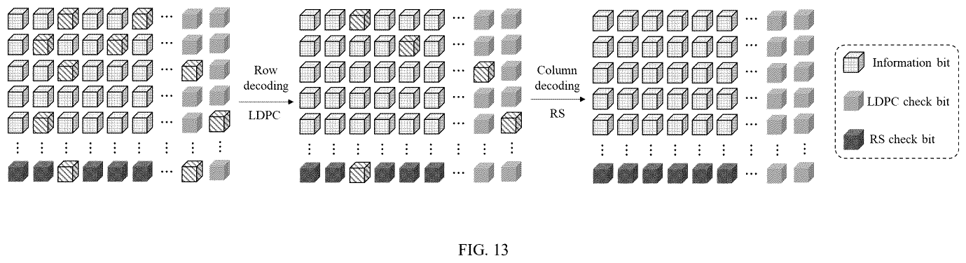

Specific steps of signal acquisition, signal analysis, incremental base calling, and primer and index identification in step (3) are as follows:
(3.1) performing real-time signal acquisition by means of the Ion Torrent sequencer, then performing signal analysis and incremental base calling in sequence, and by using a base run-length metric criterion, according to a determined reference base sequence, mapping partial sequences obtained by base calling to run-length sequences; and
(3.2) clustering the partial run-length sequences by using primers and indices, firstly, constructing reference run-length sequences of all forward and reverse primers, then calculating a distance between the run-length sequences and all reference candidates, selecting a primer label having a minimum run spatial distance, and checking whether a minimum distance is less than a threshold, to determine a most possible primer;
(3.3) after primer identification, demapping the base sequences into bit sequences, and performing de-interleaving the bit sequences, to obtain shortened Hamming codewords, finally, checking the bit sequences by using a check matrix, and if the check is correct, selecting an information bit to label the run sequences for generation of subsequent consensus sequences;
(3.4) for multiple copies of run-length sequences having same primers and indices, aligning the run-length sequences according to an offset relative to a reference run-length sequence, comparing position-by-position values of original run-length sequences with updated values by using the feedback result of successful decoding of the previous layer, and if original values are greater than the updated values, retaining the original values; otherwise, refreshing by using the updated values; and then counting number distribution of base run-lengths at different positions in the run-length sequences;
(3.5) performing position-by-position majority voting on the run-length sequences having multiple copies, to obtain consensus run-length sequences; if a voting result at a certain position is not unique, taking a base run-length corresponding to frequency suboptimality as a consensus voting result at the position; further demapping the consensus run-length sequences into partial base sequences according to a determined reference base sequence; and further placing the partial base sequences at determined positions of the coding base layers according to the determined primers and indices;
(3.6) counting and updating a base available ratio of each individual coding base layer, if the ratio reaches a preset threshold of successful decoding, e.g., 98.5%, outputting coding base layers that have not been successfully decoded;
(3.7) performing iterative decoding on the output coding base layers, wherein the specific decoding process thereof is as shown in FIG. 13, firstly, LDPC decoding is performed, the coding base layers having 10,079,472 bases are transformed into 20,158,944 bits and divided into 312 LDPC codewords, each having a length of 64,612 bits, and each LDPC codeword is decoded to obtain an information vector having a length of 54,000 bits (6,750 bytes);
(3.8) performing RS decoding in a column, and forming 13,500 RS codewords from 54,000 bit information bit vectors in all 312 encoding words and independently decoding same, wherein each RS codeword has 156 symbols in a Galois Field GF(2⁸), and each symbol consists of 4 bits at same positions of two LDPC codewords; and therefore, a total of 13,500 RS codewords are formed;
(3.9) after RS decoding, feeding back a decoding result to LDPC decoding to replace original bits, and repeating the iteration process many times, wherein the iteration process is repeated three times in this embodiment, and after a preceding coding base layer is successfully decoded, it is fed back to a next layer for decoding to assist in updating run sequences, thereby eliminating error propagation between cross layers;
(3.10) scheduling 304 data blocks subjected to product code decoding in the first layer into three stream files: an audio stream, a video stream, and control data, and then scheduling the remaining layers (layer 2 to layer 28) into audio and video stream files;
(3.11) alternately splicing sample units of an audio and a video for each data layer by using recovered control data, wherein the sample unit is a minimum unit for storing audio information and video information in an MP4 encapsulation format;
(3.12) based on the principle that audio and video samples should be performed synchronously, for any superfluous partial samples of the layer, caching data waiting for the next layer to fill a complete sample, and repeating this process until all the layer data is assembled into 28 streaming media fragments; and
(3.13) based on a first-in-first-out (FIFO) unit, setting an audio and video buffer for storing the streaming media fragments assembled from data of each layer, and when a layer is playing, temporarily storing newly decoded streaming media fragments in the buffer for subsequent playing. The playable durations and data volumes of the 28 layers of corresponding film clips actually readout in real time are as shown in Table 1.

TABLE 1
Playable durations and data volumes of film
clips actually readout in real time

| | | | | Audio | |
|---|---|---|---|---|---|
| | | | | and | |
| | | | | video | Control |
| | Start | End | Duration | data | data |
| Layer | time | time | (sec) | (byte) | (byte) |
| 1 | 00: 00.000 | 00: 29.280 | 29.28 | 898,577 | 1,543,706 |
| 2 | 00: 29.280 | 01: 35.737 | 66.46 | 1,736,705 | |
| 3 | 01: 35.737 | 02: 40.218 | 64.48 | 2,829,174 | |
| 4 | 02: 40.218 | 03: 46.675 | 66.46 | 2,046,738 | |
| 5 | 03: 46.675 | 04: 51.109 | 64.43 | 2,113,423 | / |
| 6 | 04: 51.109 | 05: 57.542 | 66.43 | 1,989,895 | |
| 7 | 05: 57.542 | 06: 59.040 | 61.50 | 2,434,921 | |
| 8 | 06: 59.040 | 08: 07.433 | 68.39 | 1,950,290 | |
| 9 | 08: 07.433 | 09: 13.867 | 66.43 | 1,990,089 | |
| 10 | 09: 13.867 | 10: 18.301 | 64.43 | 1,983,743 | |
| 11 | 10: 18.301 | 11: 24.735 | 66.43 | 2,149,109 | |
| 12 | 11: 24.735 | 12: 29.169 | 64.43 | 1,793,041 | |
| 13 | 12: 29.169 | 13: 35.602 | 66.43 | 2,315,509 | |
| 14 | 13: 35.602 | 14: 40.060 | 64.46 | 2,168,593 | |
| 15 | 14: 40.060 | 15: 45.493 | 65.43 | 1,850,529 | |
| 16 | 15: 45.493 | 16: 51.904 | 66.41 | 1,680,903 | |
| 17 | 16: 51.904 | 17: 56.361 | 64.46 | 2,258,983 | |
| 18 | 17: 56.361 | 19: 02.795 | 66.43 | 2,373,903 | |
| 19 | 19: 02.795 | 20: 05.920 | 63.13 | 2,114,456 | |
| 20 | 20: 05.920 | 21: 00.320 | 54.40 | 1,967,078 | |
| 21 | 21: 00.320 | 21: 57.320 | 57.00 | 1,986,643 | |
| 22 | 21: 57.320 | 23: 17.520 | 80.20 | 2,160,225 | |
| 23 | 23: 17.520 | 24: 26.960 | 69.44 | 2,086,253 | |
| 24 | 24: 26.960 | 25: 34.398 | 67.44 | 1,780,982 | |
| 25 | 25: 34.398 | 26: 40.878 | 66.48 | 1,483,535 | |
| 26 | 26: 40.878 | 27: 45.312 | 64.43 | 1,604,941 | |
| 27 | 27: 45.312 | 28: 51.769 | 66.46 | 1,911,813 | |
| 28 | 28: 51.769 | 29: 59.594 | 67.82 | 2,248,877 | |
| Total | | / | 1,799.59 | 57,452,634 | |

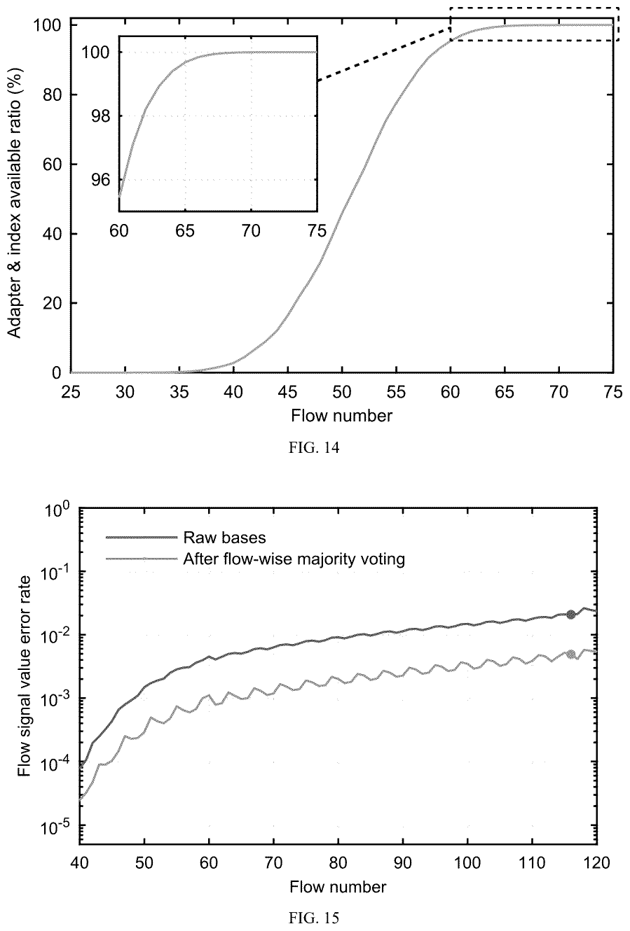

To verify that the base run-length metric criterion based on the determined sequence provided by the present disclosure can assist in rapid read-out in a scenario of the presence of a homopolymer, TACG is used as a determined reference sequence. FIG. 14 shows a diagram of cumulative probability distribution between run-length sequence length distribution and base available ratios obtained by using the statistic of 10,079,472 sequences including 12 groups of different primers and 419,978 index sequences, and it can be seen that the cumulative probability distribution has a longer tailing, and even if the sequences in the tailing portion in the diagram are discarded, up to 99% of the sequences can still be recovered.

FIG. 15 shows a comparison diagram of error rate performance at a signal level before and after majority voting of multi-copy signals provided by the present disclosure, wherein the length of the run sequence is 120, and it can be seen that the error rate of signals at different positions is significantly reduced after majority voting of multi-copy signals is used. Table 2 shows the error rate of original base sequences and the error rate of base sequences after majority voting of multi-copy signals is used, and it can be seen that insertion/deletion errors are significantly reduced, indicating that the method provided by the present disclosure has good performance for eliminating the insertion/deletion errors.

TABLE 2
Base error rate performance comparison

| | | Majority |
|---|---|---|
| | | voting |
| | | of |
| | Original | multi-copy |
| | base | signals |
| Insertion | 0.0039 | 0.0012 |
| Deletion | 0.0012 | 0.0005 |
| Substitution | 0.0010 | 0.0003 |
| Erasing | / | 0.0008 |
| Total | 0.0061 | 0.0028 |

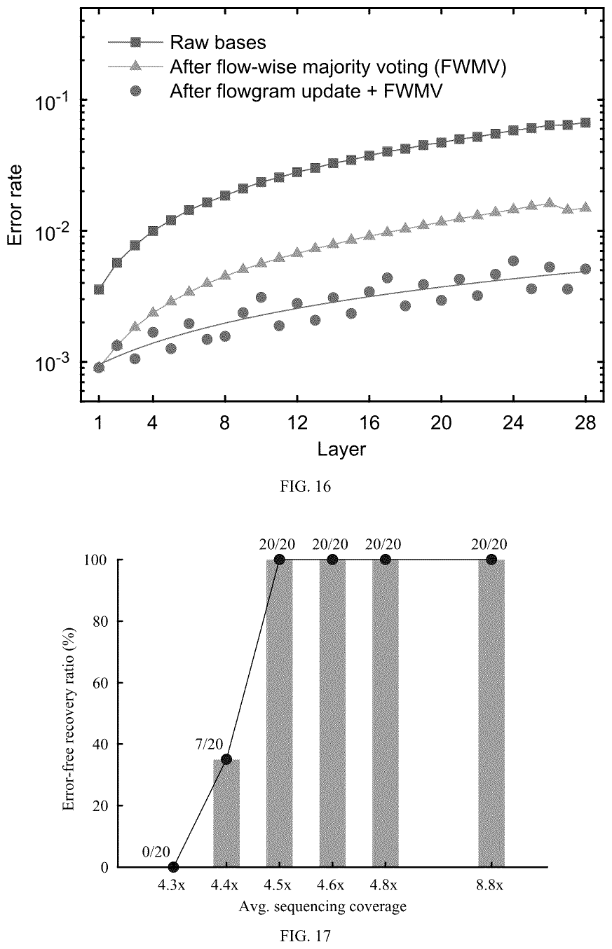

FIG. 16 shows a performance comparison diagram of majority voting of multi-copy signals provided by the present disclosure and feedback update, and it can be seen that after majority voting of multi-copy signals and feedback iterative update are used, the base error rates corresponding to the 28 layers are significantly reduced, further verifying the performance superiority of the method provided by the present disclosure.

FIG. 17 shows recovery of data under different sequencing coverage, and it can be seen that under a low sequencing coverage of 4.5×, error-free recovery of 28 layers of data can be achieved, proving that the DNA storage method provided by the present disclosure can achieve reliable storage read-out of data under a low sequencing coverage.

Person skilled in the art may understand that the drawings are merely schematic diagrams of a preferred embodiment, and the serial numbers of the foregoing embodiments of the present disclosure are merely for description, and do not represent the preference of the embodiments.

The above descriptions are only preferred examples of the present disclosure and are not intended to limit the present disclosure. Any modifications, equivalent replacements, improvements, etc. made within the spirit and principles of the present disclosure shall be included within the scope of protection of the present disclosure.

What is claimed is:

1. A spatially layered DNA storage method for large-scale oligo pools, includes the following steps:
(1) grouping user data into L data layers having a regular length of K bits, performing error correction encoding on each individual data layer respectively to obtain an encoded data layer having a length of N bits, wherein N>K, and according to a determined mapping rule between bit pairs and bases, transcoding the encoded data layer to obtain a coding base layer;
(2) according to different sequencing read-out modes, sequentially allocating bases in the coding base layer to positions from 1 to L of a DNA sequence, to constitute a payload part of a single-end read DNA sequence having a length of L, and sequentially allocating the coding base layer to symmetrical positions at two ends of the DNA sequence, to obtain a payload part of a paired-end read DNA sequence having a length of 2L;
(3) adding indices and primers to generated DNA data bearing sequences, to obtain large-scale oligonucleotide DNA sequences;
(4) performing sequencing library preparation on each oligo pool obtained by synthesis, and further, performing simultaneously sequencing and readout via a high-throughput sequencing technology for aligning and immobilizing oligonucleotides on a solid-phase carrier;
(5) subjecting oligonucleotide sequences aligned and immobilized on a surface of the solid-phase carrier to real-time optical signal or electrical signal acquisition, signal analysis, incremental base calling, and primer and index identification, and when performing depolymerization detection by using naturally unmodified deoxy-ribonucleoside triphosphate (dNTP), obtaining partial run-length sequences in a base run-length metric form, wherein the base run-length represents a length of a continuous base obtained by recognition when a current nucleotide is used for polymerization; and when performing depolymerization detection by using modified dNTP with a terminator and a fluorescent group, obtaining a presence or absence of a signal by optical detection, i.e., judging a presence or absence of a single base;
(6) according to the identified primers and indices, obtaining multi-copy signals in different base signal forms, clustering the signals, then performing multi-copy merging of the signals, and transforming same to generate consensus run-length sequences;
(7) according to whether there are insertion and deletion errors during sequencing, setting a run-length sequence feedback update mechanism, and in a case where there are no insertion and deletion errors, directly transforming the consensus run-length sequences into coding base layers; and in a case where there is insertion and deletion error propagation, updating the partial run-length sequences by using a feedback result of successful decoding of a previous layer, generating consensus run-length sequences by using multi-copy majority voting, then transforming consensus t partial run-length sequences into partial base sequences according to a determined reference base sequence, and sequentially allocating the partial base sequences to determined positions of the coding base layers, and forming individual coding base layers by bases at same positions; and
(8) counting and updating base available ratios of all the coding base layers, performing threshold comparison, outputting the coding base layers that are not successfully decoded, sending same to a decoder for decoding, and recovering original user data layer by layer.

2. The spatially layered DNA storage method for large-scale oligo pools according to claim 1, wherein the grouping user data into L data layers having a regular length of K, performing error correction encoding on each individual data layer respectively to obtain an encoded data layer having a length of N bits, wherein N>K, and according to a determined mapping rule between bit pairs and bases, transcoding the encoded data layer to obtain a coding base layer, has the following specific steps:
(1.1) averagely dividing user data into L groups that correspond to L data layers, wherein a size of each layer of data is K bits;
(1.2) scrambling individual data layers by superposing same-length pseudorandom sequences, and encoding same by using a linear block code (N, K), to obtain encoded data layers having a size of N bits; and traversing all the L data layers, and repeatedly executing above operations, to obtain L encoded data layers; and
(1.3) according to the determined mapping rule between bit pairs and bases, i.e., {00→A, 01→T, 10→G, 11→C}, transcoding the L encoded data layers respectively to obtain L coding base layers.

3. The spatially layered DNA storage method for large-scale oligo pools according to claim 1, wherein the according to different sequencing read-out modes, sequentially allocating bases in the coding base layer to positions from 1 to L of a DNA sequence, to constitute a payload part of a single-end read DNA sequence having a length of L, and sequentially allocating the coding base layer to symmetrical positions at two ends of the DNA sequence, to obtain a payload part of a paired-end read DNA sequence having a length of 2L, has the following specific steps:
(2.1) taking out one base in sequence from same positions of the L coding base layers, and allocating same in sequence to positions from 1 to L of a data DNA sequence; and traversing all positions of the coding base layers, and repeatedly executing same steps, to obtain N/2 single-end read DNA sequences having a length of L bases, wherein a base length d of an oligonucleotide sequence constructing a payload part satisfies 20≤d≤500; and
(2.2) taking out two bases respectively from same positions of the L coding base layers, and according to a basic criterion that a first layer of bases is located outside a sequence and a last layer of bases is located inside the sequence, splicing base pairs to same positions at two ends of a symmetrical DNA sequence, respectively, to constitute a payload part of a single paired-end read DNA sequence having a length of 2L bases; and traversing all positions of the coding base layers, and repeatedly executing same steps, to obtain N/4 paired-end read data DNA sequences having a length of 2L bases, wherein a base length d of an oligonucleotide sequence constructing a payload part satisfies 20≤d≤500.

4. The spatially layered DNA storage method for large-scale oligo pools according to claim 1, wherein the adding indices and primers to generated DNA data bearing sequences, to obtain large-scale oligonucleotide DNA sequences, is specifically as follows: adding a unique index having a base length of n to two ends of each DNA sequence, respectively, wherein different oligo pools share a same group of indices, further adding a fixed-length primer to a single end/paired ends of DNA sequences, to construct complete oligo pools, and performing high-throughput synthesis.

5. The spatially layered DNA storage method for large-scale oligo pools according to claim 1, wherein the performing sequencing library preparation on each oligo pool obtained by synthesis, performing read-out by sequencing by means of a high-throughput sequencing technology for aligning and immobilizing oligonucleotides on a solid-phase carrier, is specifically as follows:
(3.1) corresponding to paired-end symmetric mapping construction of oligonucleotide sequences of the coding base layers, by using a standard library preparation method, performing polymerase chain reaction amplification on the synthetic oligo pools by using forward and reverse primer pairs, respectively, taking out a small number of samples for adding sequencing adapters, and constructing a sequencing library which is loaded to a sequencer for sequencing;
(3.2) corresponding to paired-end symmetric mapping construction of oligonucleotide sequences of the coding base layers, by using a bidirectional library preparation method, performing polymerase chain reaction amplification on the oligo pools by using paired forward and reverse overhanging primers, respectively, to obtain two sequencing libraries which are then mixed, and taking out a small number of samples for single-template amplification on a solid-phase carrier, including an overhanging primer complementary sequence, to complete preparation of sequencing libraries which are loaded to a sequencer for sequencing; and
(3.3) corresponding to single-end asymmetric mapping construction of oligonucleotide sequences of the coding base layers, by using a single-end library preparation method, performing polymerase chain reaction amplification on the synthetic oligo pools only by using a forward primer by using fixed amplification in one direction, taking out a small number of samples for single-template amplification on a solid-phase carrier, including an overhanging primer complementary sequence, to complete preparation of sequencing libraries which are loaded to a sequencer for sequencing.

6. The spatially layered DNA storage method for large-scale oligo pools according to claim 1, wherein the subjecting oligonucleotide sequences aligned and immobilized on a surface of the solid-phase carrier to real-time optical signal or electrical signal acquisition, signal analysis, incremental base calling, and primer and index identification, to obtain partial run-length sequences in a base run-length metric form, has the following specific steps:
(4.1) during incremental base calling, recording base calling results in real time, and by using a base run-length metric criterion, according to a determined reference base sequence, mapping partial base sequences obtained by base calling to partial run-length sequences; and
(4.2) performing primer identification on the identified partial run-length sequences by using a run space distance metric, determining a boundary thereof, starting from a primer recognition boundary, intercepting the partial run-length sequences, demapping same into base sequences having a same length as that of an index part, pre-processing the index part, performing validity checking or error correction by using a check matrix to retain an information part of valid partial run sequences, and labeling corresponding primers, labels and relative start offset from a reference run sequence.

7. The spatially layered DNA storage method for large-scale oligo pools according to claim 1, wherein the according to the identified primers and indices, obtaining multi-copy signals in different base signal forms, clustering the signals, then performing multi-copy merging of the signals, and transforming same to generate consensus run-length sequences, has the following specific steps:
(5.1) counting one by one a base run-length metric value corresponding to each run-length sequence of the multi-copy signals;
(5.2) executing majority voting, and outputting a most frequent base run-length metric; and
(5.3) transforming into a base sequence according to the base run-length metric value, and outputting layer by layer a base sequence after multi-copy merging.

8. The spatially layered DNA storage method for large-scale oligo pools according to claim 1, wherein the according to whether there are insertion and deletion errors during sequencing, setting a run-length sequence feedback update mechanism, and in a case where there are no insertion and deletion errors, directly transforming the consensus run-length sequences into coding base layers; and in a case where there is insertion and deletion error propagation, updating the partial run-length sequences by using a feedback result of successful decoding of a previous layer, generating consensus run-length sequences by using multi-copy majority voting, then transforming consensus partial run-length sequences into partial base sequences according to a determined reference base sequence, and sequentially allocating the partial base sequences to determined positions of the coding base layers, and forming individual coding base layers by bases at same positions, has the following specific steps:
(6.1) updating multiple copies of partial run-length sequences having same primers and indices by using the feedback result of successful decoding of the previous layer, then performing majority voting position by position, generating consensus partial run-length sequences, demapping the consensus partial run-length sequences into partial base sequences according to a determined reference base sequence, and sequentially placing the partial base sequences at determined positions of the L coding base layers according to the identified primers and indices, wherein each coding base layer includes N/2 unique sites;
(6.2) counting and updating a base available ratio of each individual coding base layer, if the ratio reaches a preset threshold of successful decoding, outputting coding base layers that have not been successfully decoded, further, transforming the output coding base layers into bits, performing error correction by using the linear block code, and recovering and obtaining user data corresponding to current data layers; and
(6.3) generating ideal partial base sequences by using coding base layers that are successfully decoded previously, transforming same according to the determined reference base sequence into ideal partial run-length sequences, generating total N/2 partial run-length sequences, and feeding back the partial run sequences generated again to steps (5.1) to (5.3) to re-execute majority voting.

9. The spatially layered DNA storage method for large-scale oligo pools according to claim 1, wherein the counting and updating base available ratios of all the coding base layers, performing threshold comparison, outputting the coding base layers that are not successfully decoded, sending same to a decoder for decoding, and recovering original user data layer by layer, has the following specific steps:
(7.1) transforming the generated coding base layers after merging into bits, and decoding same by using the linear block code for error correction; and
(7.2) sequentially executing merging and decoding of each layer of data until all layers of data are completely read out, to achieve real-time DNA storage readout through simultaneous sequencing and decoding.

10. The spatial stratification DNA storage method for large-scale oligo pools according to claim 2, wherein the scrambling individual data layers by superposing same-length pseudorandom sequences, and encoding same by using a linear block code (N, K), to obtain encoded data layers having a size of N bits; and traversing all the L data layers, and repeatedly executing same operations, to obtain L encoded data layers, has the following specific steps:
(8.1) encoding data by a linear block code using a product code, and dividing each layer of data into P data blocks having a same size;
(8.2) executing an encoding process of a first component code in product code encoding on P data blocks in each layer, adding a check data block, and generating M data blocks, to constitute codeword blocks of the first component code of a single layer;
(8.3) executing an encoding process of a second component code in product code encoding, encoding each data block in each layer into the codeword of the second component code by using a generation matrix, and generating total M codewords of the second component code in each layer; and
(8.4) traversing all the L data layers, and repeatedly executing a product code encoding process, to finally obtain L encoded data layers.

11. The spatially layered DNA storage method for large-scale oligo pools according to claim 4, wherein the adding indices and primers to generated DNA data bearing sequences, to obtain large-scale oligonucleotide DNA sequences, being specifically as follows: adding a unique index having a base length of n to two ends of each DNA sequence, respectively, wherein different oligo pools share a same group of indices, further adding a fixed-length primer to a single end/paired ends of DNA sequences, to construct complete oligo pools, and performing high-throughput synthesis, has the following specific steps:
(9.1) traversing all natural numbers within a range of [0, 2^{k}], representing same by using bit vectors having a length of k bits, encoding each bit vector by using a binary short block error correction code (n, k), and randomly interleaving obtained codewords, wherein, k≥┌log₂\hat{N}┐ and is a positive integer, \hat{N} represents the number of base sequences included in the oligo pools, and ┌g┐ represents rounding up to an integer;
(9.2) transcoding encoded codeword sequences according to a determined mapping rule to obtain index sequences having a base length of n/2, counting sequence homopolymer length distribution, according to a minimum homopolymer length criterion, preferentially selecting a base sequence having a smaller homopolymer length, and for single-end read DNA sequences, screening N/2 sequences as valid indices; and for paired-end read DNA sequences, screening N/4 sequences as valid indices; and
(9.3) adding the index base sequences to forward ends of data DNA sequences, then randomly interleaving index DNA sequences at a base level, and adding same to the other ends of the data DNA sequences, to generate DNA sequences where index identification can be performed at two ends.

12. The spatially layered DNA storage method for large-scale oligo pools according to claim 6, wherein the performing primer identification on the identified partial run-length sequences by using a run space distance metric, determining a boundary thereof, starting from a primer recognition boundary, intercepting the partial run-length sequences, demapping same into base sequences having a same length as that of an index part, pre-processing the index part, performing validity checking or error correction by using a check matrix to retain an information part of valid partial run-length sequences, and labeling corresponding primers, indices and relative start offset from a reference run sequence, has the following specific steps:
(10.1) by using a known reference base sequence, constructing reference run-length sequences of all forward and reverse primers used in the oligo pools, comparing partial run-length sequences identified after accumulation during real-time base calling with the reference run-length sequences to obtain a spatial distance, and determining primer classifications to which corresponding partial sequences belong;
(10.2) searching for a primer having a minimum run spatial distance according to a comparison result, comparing the run spatial distance thereof with a preset threshold, if the preset threshold is satisfied, considering that the primer is valid, and retaining a corresponding sequence and labeling a most possible primer; otherwise, discarding the corresponding run-length sequence;
(10.3) demapping index part of base sequences to bit sequences, and deinterleaving same to obtain codewords corresponding to the indices; and
(10.4) performing validity checking by using a check matrix, if checking is correct and the indices are legal, considering that the indices are valid, and retaining run-length sequences of corresponding information part; and performing error correction on index part by using a short block error correction code, recovering original indices, checking index legality, and retaining run-length sequences of corresponding information part.

13. The spatially layered DNA storage method for large-scale oligo pools according to claim 8, wherein the updating multiple copies of partial run-length sequences having same primers and indices by using the feedback result of successful decoding of the previous layer, then performing majority voting position by position, generating consensus partial run-length sequences, demapping the consensus partial run-length sequences into partial base sequences according to a determined reference base sequence, and sequentially placing the partial base sequences at determined positions of the L coding base layers according to the identified primers and indices, wherein each coding base layer includes N/2 unique sites, has the following specific steps:
(11.1) for multiple copies of partial run-length sequences having same primers and indices, aligning the partial run-length sequences according to a start offset relative to a reference run-length sequence, comparing position-by-position values of original run-length sequences with updated values by using the feedback result of successful decoding of the previous layer, and if original values are greater than the updated values, retaining the original values; otherwise, refreshing by using the updated values;
(11.2) performing position-by-position majority voting on the partial run-length sequences having multiple copies, to obtain consensus partial run-length sequences; and if a voting result at a certain position is not unique, taking a base run-length corresponding to frequency suboptimality as a consensus voting result at the position; and
(11.3) demapping the consensus partial run-length sequences into partial base sequences according to a determined reference base sequence, and placing the partial base sequences at determined positions of the coding base layers according to the determined primers and indices.
